(19) United States
(12) Patent Application Publication
(10) Pub. No.: US 2007/0105887 A1
(43) Pub. Date: May 10, 2007

(54) Antineoplastic combinations of temsirolimus and sunitinib malate

(75) Inventor: Laurence Moore, Newton, MA (US)

Correspondence Address: HOWSON AND HOWSON;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/591,979

(22) Filed: Nov. 2, 2006

Related U.S. Application Data
(60) Provisional application No. 60/733,564, filed on Nov. 4, 2005.

(51) Int. Cl. A61K 31/4745; A61K 31/405

(52) U.S. Cl. 514/291; 514/419

(57) ABSTRACT

A combination of temsirolimus and sunitinib malate in the treatment of cancer is provided. Also provided are regimens and kits for treatment of renal cell carcinoma, containing temsirolium and sunitinib malate, optionally in combination with other anti-neoplastic or immune modulators.

### CROSS-REFERENCE TO RELATED APPLICATION

This application claims the benefit under 35 U.S.C 119(e) of U.S. Provisional Patent Application No. 60/733,564, filed Nov. 4, 2005.

### BACKGROUND OF THE INVENTION

This invention relates to the use of combinations of an mTOR inhibitor and sunitinib malate for the treatment of neoplasms.

CCI-779, is rapamycin 42-ester with 3-hydroxy-2-(hydroxymethyl)-2-methylpropionic acid, an ester of rapamycin which has demonstrated significant inhibitory effects on tumor growth in both in vitro and in vivo models. This compound is now known generically under the name temsirolimus. The preparation and use of hydroxyesters of rapamycin, including temsirolimus, are described in U.S. Pat. Nos. 5,362,718 and 6,277,983.

Temsirolimus exhibits cytostatic, as opposed to cytotoxic properties, and may delay the time to progression of tumors or time to tumor recurrence. Temsirolimus is considered to have a mechanism of action that is similar to that of sirolimus. Temsirolimus binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from G1 to S. The mechanism of action of temsirolimus that results in the G1-S phase block is novel for an anticancer drug. Temsirolimus has been described as an agent in connection with the treatment of renal cell carcinoma, amongst others.

Sunitinib malate or SU11248, is an orally bioavailable indolinone with potential antineoplastic activity. SU11248 blocks the tyrosine kinase activities of vascular endothelial growth factor receptor 2 (VEGFR2), platelet-derived growth factor receptor β (PDGFRβ), and c-kit, thereby inhibiting angiogenesis and cell proliferation. This agent also inhibits the phosphorylation of Fms-related tyrosine kinase 3 (FLT3), another receptor tyrosine kinase expressed by some leukemic cells. This compound, sinitinib malate, is available under the registered trademark “Sutent” (Pfizer).

What is needed is an improved antineoplastic therapy.

### DETAILED DESCRIPTION OF THE INVENTION

This invention provides the use of combinations comprising an mTOR inhibitor and sunitinib malate in the treatment of neoplasms. The invention further provides products containing an mTOR inhibitor and sunitinib malate formulated for simultaneous, separate or sequential use in treating neoplasms in a mammal. The following detailed description illustrates temsirolimus. However, other mTOR inhibitors may be substituted for temsirolimus in the methods, combinations and products described herein.

These methods, combinations and products are useful in the treatment of a variety of neoplasms including, for example, renal cancer, soft tissue cancer, breast cancer, neuroendocrine tumor of the lung, cervical cancer, uterine cancer, head and neck cancer, glioma, non-small lung cell cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer. In one embodiment, the combination of temsirolimus and sunitinib malate is particularly well suited for treatment of renal cell carcinoma.

As used herein, the term mTOR inhibitor means a compound or ligand, or a pharmaceutically acceptable salt thereof, that inhibits cell replication by blocking the progression of the cell cycle from G1 to S. The term includes the neutral tricyclic compound rapamycin (sirolimus) and other rapamycin compounds, including, e.g., rapamycin derivatives, rapamycin analogues, other macrolide compounds that inhibit mTOR activity, and all compounds included within the definition below of the term “a rapamycin”. These include compounds with a structural similarity to “a rapamycin”, e.g., compounds with a similar macrocyclic structure that have been modified to enhance therapeutic benefit. FK-506 can also be used in the method of the invention.

As used herein, the term a rapamycin defines a class of immunosuppressive compounds that contain the basic rapamycin nucleus as shown below. 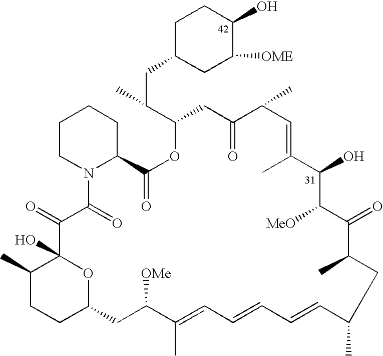
The rapamycins of this invention include compounds that are chemically or biologically modified as derivatives of the rapamycin nucleus, while still retaining immunosuppressive properties. Accordingly, the term a rapamycin includes rapamycin, and esters, ethers, carbamates, oximes, hydrazones, and hydroxylamines of rapamycin, as well as rapamycins in which functional groups on the rapamycin nucleus have been modified, for example through reduction or oxidation. Also included in the term a rapamycin are pharmaceutically acceptable salts of rapamycins.

The term a rapamycin also includes 42- and/or 31-esters and ethers of rapamycin as described in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118, 678); silyl esters (U.S. Pat. No. 5,120,842); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers is disclosed in the patents listed above.

Further included within the definition of the term a rapamycin are 27-esters and ethers of rapamycin, which are disclosed in U.S. Pat. No. 5,256,790. Also described are C-27 ketone rapamycins which are reduced to the corresponding alcohol, which is in turn converted to the corresponding ester or ether. The preparation of these esters and ethers is disclosed in the patent listed above. Also included are oximes, hydrazones, and hydroxylamines of rapamycin are disclosed in U.S. Pat. Nos. 5,373,014, 5,378,836, 5,023,264, and 5,563,145. The preparation of these oximes, hydrazones, and hydroxylamines is disclosed in the above-listed patents. The preparation of 42-oxorapamycin is disclosed in U.S. Pat. No. 5,023,263.

As used herein, the term a CCI-779 means rapamycin 42-ester with 3-hydroxy-2-(hydroxymethyl)-2-methylpropionic acid (temsirolimus), and encompasses prodrugs, derivatives, pharmaceutically acceptable salts, or analogs thereof.

Examples of a rapamycin include, e.g., rapamycin, 32-deoxorapamycin, 16-pent-2-ynyloxy-32-deoxorapamycin, 16-pent-2-ylyloxy-32(S)-dihydro-rapamycin, 16-pent-2-ylyloxy-32(S)-dihydr-o-40-O-(2-hydroxyethyl)-rapamycin, 40-O-(2-hydroxyethyl)-rapamycin, rapamycin 42-ester with 3-hydroxy-2-(hydroxymethyl)-2-methylpropionic acid (CCI-779), 40-[3-hydroxy-2-(hydroxymethyl)-2-methylpropanoate]-rapamycin, or a pharmaceutically acceptable salt thereof, as disclosed in U.S. Pat. No. 5,362,718, ABT578, or 40-(tetrazolyl)-rapamycin, 40-epi-(tetrazolyl)-rapamycin, e.g., as disclosed in International Patent Publication No. WO 99/15530, or rapamycin analogs as disclosed in International Patent Publication No. WO 98/02441 and WO 01/14387, e.g., AP23573. In another embodiment, the compound is Certican™ (everolimus, 2-O-(2-hydroxy)ethyl rapamycin, Novartis, U.S. Pat. No. 5,665,772).

The following standard pharmacological test procedure can be used to determine whether a compound is an mTOR inhibitor, as defined herein. Treatment of growth factor stimulated cells with an mTOR inhibitor like rapamycin completely blocks phosphorylation of serine 389 as evidenced by Western blot and as such constitutes a good assay for mTOR inhibition. Thus, whole cell lysates from cells stimulated by a growth factor (e.g. IGFl) in culture in the presence of an mTOR inhibitor should fail to show a band on an acrylamide gel capable of being labeled with an antibody specific for serine 389 of p70s6K.

It is preferred that the mTOR inhibitor used in the antineoplastic combinations of this invention is a rapamycin, and more preferred that the mTOR inhibitor is rapamycin, temsirolimus, or 42-O-(2-hydroxy)ethyl rapamycin. The preparation of 42-O-(2-hydroxy)ethyl rapamycin is described in U.S. Pat. No. 5,665,772.

The preparation of temsirolimus is described in U.S. Pat. No. 5,362,718. A regiospecific synthesis of temsirolimus is described in U.S. Pat. No. 6,277,983, which is hereby incorporated by reference. Still another regiospecific method for synthesis of temsirolimus is described in U.S. patent application Ser. No. 10/903,062, filed Jul. 30, 2004, US Patent Publication No. 2005-0033046-A1, publication number Feb. 10, 2005 and its counterpart, International Patent Publication No. WO 2005/016935, publication Apr. 7, 2005.

Sunitinib malate, and methods of making and formulating same have been described. See, e.g., WO 2001060814 and U.S. Pat. No. 6,573,293, and particularly, claim 49 of the WO and claim 5 of the US.

As used in accordance with this invention, the term “treatment” means treating a mammal having a neoplasm by providing said mammal an effective amount of a combination of an mTOR inhibitor and sunitinib malate with the purpose of inhibiting progression of the neoplastic disease, growth of a tumor in such mammal, eradication of the neoplastic disease, prolonging survival of the mammal and/or palliation of the mammal.

As used in accordance with this invention, the term “providing,” with respect to providing an mTOR inhibitor and sunitinib malate, means either directly administering the mTOR inhibitor, or administering a prodrug, derivative, or analog which will form an effective amount of the mTOR inhibitor within the body, along with sunitinib malate directly, or administering a prodrug, derivative, or analog which will form an effective amount of sunitinib malate in the body.

Use of a combination of an MTOR inhibitor (e.g., temsirolimus) and sunitinib malate also provides for the use of combinations of each of the agents in which one or both of the agents is used at subtherapeutically effective dosages. Subtherapeutically effective dosages may be readily determined by one of skill in the art, in view of the teachings herein. In one embodiment, the subtherapeutically effective dosage is a dosage which is effective at a lower dosage when used in the combination regimen of the invention, as compared to the dosage that is effective when used alone. The invention further provides for one or more of the active agents in the combination of the invention to be used in a supratherapeutic amount, i.e., at a higher dosage in the combination than when used alone. In this embodiment, the other active agent(s) may be used in a therapeutic or subtherapeutic amount.

The combinations of the invention may be in the form of a kit of parts. The invention therefore includes a product containing an mTOR inhibitor and sunitinib malate as a combined preparation for simultaneous, separate or sequential use in treating a neoplasm in a mammal in need thereof. In one embodiment, a product contains temsirolimus and sunitinib malate as a combined preparation for simultaneous, separate or sequential use in treating renal cell carcinoma in a mammal in need thereof.

The invention also includes a pharmaceutical pack containing a course of treatment of a neoplasm for one individual mammal, wherein the pack contains units of an mTOR inhibitor in unit dosage form and units of sunitinib malate in unit dosage form. In one embodiment, a pharmaceutical pack contains a course of treatment of renal cell carcinoma for one individual mammal, wherein the pack contains units of temsirolimus in unit dosage form and units of sunitinib malate in unit dosage form.

Administration of the compositions may be oral, intravenous, respiratory (e.g., nasal or intrabronchial), parenteral (besides i.v., such as intraperitoneal and subcutaneous injections), intraperitoneal, transdermal (including all administration across the surface of the body and the inner linings of bodily passages including epithelial and mucosal tissues), and vaginal (including intrauterine administration). Other routes of administration are also feasible, such as via implants, rectally, intranasally.

While the components of the invention may be delivered via the same route, a product or pack according to the invention may contain an mTOR inhibitor, such as temsirolimus, for delivery by a different route than that of the sunitinib malate, e.g., one component may be delivered orally, while the other is administered intravenously. In one embodiment, temsirolimus is prepared for intravenous delivery and sunitinib malate is prepared for oral delivery. In another embodiment, temsirolimus and sunitinib malate are both delivered by the same route, e.g., orally or i.v. Other variations would be apparent to one skilled in the art and are contemplated within the scope of the invention.

As is typical with oncology treatments, dosage regimens are closely monitored by the treating physician, based on numerous factors including the severity of the disease, response to the disease, any treatment related toxicities, age, and health of the patient. It is projected that initial i.v. infusion dosages of the mTOR inhibitor (e.g., temsirolimus) will be from about 5 to about 175 mg, or about 5 to about 25 mg, when administered on a weekly dosage regimen. Other dosage regimens and variations are foreseeable, and will be determined through physician guidance. It is preferred that the mTOR inhibitor is administered by i.v. infusion or orally, preferably in the form of tablets or capsules.

For sunitinib malate, single doses and multiple doses are contemplated. In one embodiment, single doses are provided orally at concentrations of from 10 to 100 mg daily, or about 12.5 to 50 mg daily. Typically, sunitinib malate is delivered for two, three, four or more consecutive weekly doses followed by a period of about 1 or 2 weeks, or more where no sunitinib malate is delivered. In one embodiment, the doses are delivered for about 4 weeks, with 2 weeks off. In another embodiment, the sunitinib malate is delivered orally for two weeks, with 1 week off. These regimens may be repeated, or alternated, as desired. Other dosage regimens and variations are foreseeable, and will be determined through physician guidance.

As described herein, subtherapeutically effective amounts of sunitinib malate and temsirolimus may be used to achieve a therapeutic effect when administered in combination. For example, sunitinib malate may be provided at dosages of 5 to 50% lower, 10 to 25% lower, or 15 to 20% lower, when provided along with temsirolimus. For example, a resulting sunitinib malate dosage can be from about 8 to 40 mg, or about 8 to 30 mg, or 8 to 25 mg. Subtherapeutically effective amounts of sunitinib malate are expected to reduce the side-effects of sunitinib malate treatment.

Dosage regimens are expected to vary according to the route of administration. It is projected that the oral dosage of an mTOR useful in the invention will be 10 mg/week to 250 mg/week, about 20 mg/week to about 150 mg/week, about 25 mg/week to about 100 mg/week, or about 30 mg/week to about 75 mg/week. For rapamycin, the projected oral dosage will be between 0.1 mg/day to 25 mg/day. Precise dosages will be determined by the administering physician based on experience with the individual subject to be treated.

Oral formulations containing the mTOR inhibitor (and optionally, other active compounds) useful in this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed. Preferred oral formulations for rapamycin 42-ester with 3-hydroxy-2-(hydroxymethyl)-2-methylpropionic acid are described in US Patent Publication No. 2004/0077677 A1, published Apr. 22, 2004.

In some cases it may be desirable to administer the compounds directly to the airways in the form of an aerosol.

The compounds may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils. Preferred injectable formulations for rapamycin 42-ester with 3-hydroxy-2-(hydroxymethyl)-2-methylpropionic acid are described in US Patent Publication No. 2004/0167152 A1, published Aug. 26, 2004.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The mTOR inhibitor plus sunitinib malate combination may be administered as the sole active antineoplastic agents. Alternatively, the mTOR inhibitor/sunitinib malate combination is part of a regimen with other active agents, including, e.g., chemotherapeutic agents, such as alkylating agents; hormonal agents (i.e., estramustine, tamoxifen, toremifene, anastrozole, or letrozole); antibiotics (i.e., plicamycin, bleomycin, mitoxantrone, idarubicin, dactinomycin, mitomycin, or daunorubicin); antimitotic agents (i.e., vinblastine, vincristine, teniposide, or vinorelbine); topoisomerase inhibitors (i.e., topotecan, irinotecan, etoposide, or doxorubicin); and other agents (i.e., hydroxyurea, trastuzumab, altretamine, rituximab, paclitaxel, docetaxel, L-asparaginase, or gemtuzumab ozogamicin); biochemical modulating agents, imatib, EGFR inhibitors such as EKB or other multi-kinase inhibitors, e.g., those that targets serine/threonine and receptor tyrosine kinases in both the tumor cell and tumor vasculature, or immunomodulators (i.e., interferons, IL-2, or BCG). Examples of suitable interferons include interferon α, interferon β, interferon γ, and mixtures thereof.

In one embodiment, the combination of an mTOR inhibitor and sunitinib malate may be further combined with antineoplastic alkylating agents, e.g., those described in US 2002-0198137A1. Antineoplastic alkylating agents are roughly classified, according to their structure or reactive moiety, into several categories which include nitrogen mustards, such as MUSTARGEN (meclorethamine), cyclophosphamide, ifosfamide, melphalan, and chlorambucil; azidines and epoxides, such as thiotepa, mitomycin C, dianhydrogalactitol, and dibromodulcitol; alkyl sulfinates, such as busulfan; nitrosoureas, such as bischloroethylnitrosourea (BCNU), cyclohexyl-chloroethyinitrosourea (CCNU), and methylcyclohexylchloroethylnitrosourea (MeCCNU); hydrazine and triazine derivatives, such as procarbazine, dacarbazine, and temozolomide; streptazoin, melphalan, chlorambucil, carmustine, methclorethamine, lomustine)and platinum compounds. Platinum compounds are platinum containing agents that react preferentially at the N7 position of guanine and adenine residues to form a variety of monofunctional and bifunctional adducts. (Johnson S. W., Stevenson J. P., O'Dwyer P. J. Cisplatin and Its Analogues. In Cancer Principles & Practice of Oncology 6^{th} Edition. ed. DeVita V. T., Hellman S., Rosenberg S. A. Lippincott Williams & Wilkins. Philadelphia 2001. p. 378.) These compounds include cisplatin, carboplatin, platinum IV compounds, and multinuclear platinum complexes.

The following are representative examples of alkylating agents of this invention. Meclorethamine is commercially available as an injectable (MUSTARGEN). Cyclophosphamide is commercially available as an injectable (cyclophosphamide, lyophilized CYTOXAN, or NEOSAR) and in oral tablets (cyclophosphamide or CYTOXAN). Ifosfamide is commercially available as an injectable (IFEX). Melphalan is commercially available as an injectable (ALKERAN) and in oral tablets (ALKERAN). Chlorambucil is commercially available in oral tablets (LEUKERAN). Thiotepa is commercially available as an injectable (thiotepa or THIOPLEX). Mitomycin is commercially available as an injectable (mitomycin or MUTAMYCIN). Busulfan is commercially available as an injectable (BUSULFEX) and in oral tablets (MYLERAN). Lomustine (CCNU) is commercially available in oral capsules (CEENU). Carmustine (BCNU) is commercially available as an intracranial implant (GLIADEL) and as an injectable (BICNU). Procarbazine is commercially available in oral capsules (MATULANE). Temozolomide is commercially available in oral capsules (TEMODAR). Cisplatin is commercially available as an injectable (cisplatin, PLATINOL, or PLATINOL-AQ). Carboplatin is commercially available as an injectable (PARAPLATIN).

In another embodiment, a combination of the invention may further include treatment with an antineoplastic antimetabolite, such as is described in US Patent Publication No. US 2005-0187184A1 or US 2002-0183239 A1. As used in accordance with this invention, the term “antimetabolite” means a substance which is structurally similar to a critical natural intermediate (metabolite) in a biochemical pathway leading to DNA or RNA synthesis which is used by the host in that pathway, but acts to inhibit the completion of that pathway (i.e., synthesis of DNA or RNA). More specifically, antimetabolites typically function by (1) competing with metabolites for the catalytic or regulatory site of a key enzyme in DNA or RNA synthesis, or (2) substitute for a metabolite that is normally incorporated into DNA or RNA, and thereby producing a DNA or RNA that cannot support replication. Major categories of antimetabolites include (1) folic acid analogs, which are inhibitors of dihydrofolate reductase (DHFR); (2) purine analogs, which mimic the natural purines (adenine or guanine) but are structurally different so they competitively or irreversibly inhibit nuclear processing of DNA or RNA; and (3) pyrimidine analogs, which mimic the natural pyrimidines (cytosine, thymidine, and uracil), but are structurally different so thy competitively or irreversibly inhibit nuclear processing of DNA or RNA.

The following are representative examples of antimetabolites of this invention. 5-Fluorouracil (5-FU; 5-fluoro-2,4(1H,3H)-pyrimidinedione) is commercially available in a topical cream (FLUOROPLEX or EFUDEX), a topical solution (FLUOROPLEX or EFUDEX), and as an injectable containing 50 mg/mL 5-fluorouracil (ADRUCIL or flurouracil). Floxuradine (2′-deoxy-5-fluorouridine) is commercially available as an injectable containing 500 mg/vial of floxuradine (FUDR or floxuradine). Thioguanine (2-amino-1,7-dihydro-6-H-purine-6-thione) is commercially available in 40 mg oral tablets (thioguanine). Cytarabine (4-amino-1-(beta)-D-arabinofuranosyl-2(1H)-pyrimidinone) is commercially available as a liposomal injectable containing 10 mg/mL cytarabine (DEPOCYT) or as a liquid injectable containing between 1 mg-1 g/vial or 20 mg/mL (cytarabine or CYTOSAR-U). Fludarabine (9-H-Purin-6-amine,2-fluoro-9-(5 -O-phosphono-(beta)-D-a-rabinofuranosyl) is commercially available as a liquid injectable containing 50 mg/vial (FLUDARA). 6-Mercaptopurine (1,7-dihydro-6H-purine-6-thione) is commercially available in 50 mg oral tablets (PURINETHOL). Methotrexate (MTX; N-[4-[[(2,4-diamino-6-pteridinyl)methyl]methylamino]benzoyl]-L-glutamic acid) is commercially available as a liquid injectable containing between 2.5-25 mg/mL and 20 mg-1 g/vial (methotrexate sodium or FOLEX) and in 2.5 mg oral tablets (methotrexate sodium). Gemcitabine (2′-deoxy-2′,2′-difluorocytidine monohydrochloride ((beta)-isomer)), is commercially available as a liquid injectable containing between 200 mg-1 g/vial (GEMZAR). Capecitabine (5′-deoxy-5-fluoro-N-[(pentyloxy)carbonyl]-cytidine) is commercially available as a 150 or 500 mg oral tablet (XELODA). Pentostatin ((R)-3-(2-deoxy-(beta)-D-erythro-pentofuranosyl)-3,6,7,-8-tetrahydroimidazo[4,5-d][1,3]diazepin-8-ol) is commercially available as a liquid injectable containing 10 mg/vial (NIPENT). Trimetrexate (2,4-diamino-5-methyl-6-[(3,4,5-trimethoxyanilino)methyl]quinazoline mono-D-glucuronate) is commercially available as a liquid injectable containing between 25-200 mg/vial (NEUTREXIN). Cladribine (2-chloro-6-amino-9-(2-deoxy-(beta)-D-erythropento-furanosyl)purine) is commercially available as a liquid injectable containing 1 mg/mL (LEUSTATIN).

The term “biochemical modulating agent” is well known and understood to those skilled in the art as an agent given as an adjunct to anti-cancer therapy, which serves to potentate its antineoplastic activity, as well as counteract the side effects of the active agent, e.g., an antimetabolite. Leucovorin and levofolinate are typically used as biochemical modulating agents for methotrexate and 5-FU therapy. Leucovorin (5-formyl-5,6,7,8-tetrahydrofolic acid) is commercially available as an injectable liquid containing between 5-10 mg/mL or 50-350 mg/vial (leucovorin calcium or WELLCOVORIN) and as 5-25 mg oral tablets (leucovorin calcium). Levofolinate (pharmacologically active isomer of 5-formyltetrahydrofolic acid) is commercially available as an injectable containing 25-75 mg levofolinate (ISOVORIN) or as 2.5-7.5 mg oral tablets (ISOVORIN).

In one embodiment, the regimen further comprises administration of an interferon (IFN). In this embodiment, the regimen may include, e.g., a regimen including delivery of IFN-α. Suitable doses of IFN may be readily determined by one of skill in the art. IFN may be delivered intravenously or by another suitable route, e.g. subcutaneously or intramuscularly, at a dose of, e.g., 3 to 18 MIU/3x/week. In other embodiments and route of delivery, doses of WFN may be in the range of 10 to 30 mg/week, or about 15 mg/week.

In another embodiment, the combination of the invention further includes an active agent selected from among a kinase inhibitor. Particularly desirable are multi-kinase inhibitors target serine/threonine and receptor tyrosine kinases in both the tumor cell and tumor vasculature. Examples of suitable kinase inhibitors are Sorafenib (BAY 43-9006, Bayer), which has been granted Fast Track status by the FDA for metastic renal cell cancer. Another suitable farnesyltransferase inhibitor is Zarnestra (R115777, tipifarnib). Still other suitable compounds that target Ras/Raf/MEK and/or MAP kinases include, e.g., avastin, ISIS 5132, and MEK inhibitors such as CI-1040 or PD 0325901.

As used in this invention, the combination regimen can be given simultaneously or can be given in a staggered regimen, with the mTOR inhibitor being given at a different time during the course of chemotherapy than the sunitinib malate. This time differential may range from several minutes, hours, days, weeks, or longer between administration of the at least two agents. Therefore, the term combination (or combined) does not necessarily mean administered at the same time or as a unitary dose, but that each of the components are administered during a desired treatment period. The agents may also be administered by different routes.

Pharmaceutical Packs/Kits:

The invention includes a product or pharmaceutical pack containing a course of an anti-neoplastic treatment for one individual mammal comprising one or more container(s) having one, one to four, or more unit(s) of an mTOR inhibitor (e.g., temsirolimus) in unit dosage form and, optionally, one, one to four, or more unit(s) of sunitinib malate, and optionally, another active agent.

In another embodiment, pharmaceutical packs contain a course of anti-neoplastic treatment for one individual mammal comprising a container having a unit of a rapamycin in unit dosage form, a containing having a unit of sunitinib malate, and optionally, a container with another active agent. In other embodiments, the rapamycin is rapamycin, an ester (including a 42-ester, ether (including a 42-ether), oxime, hydrazone, or hydroxylamine of rapamycin. In another embodiment, the rapamycin is 42-O-(2-hydroxy)ethyl rapamycin.

In another embodiment, the rapamycin is temsirolimus, and the pack contains one or more container(s) comprising one, one to four, or more unit(s) of temsirolimus with the components described herein.

In some embodiments, the compositions of the invention are in packs in a form ready for administration. In other embodiments, the compositions of the invention are in concentrated form in packs, optionally with the diluent required to make a final solution for administration. In still other embodiments, the product contains a compound useful in the invention in solid form and, optionally, a separate container with a suitable solvent or carrier for the compound useful in the invention.

In still other embodiments, the above packs/kits include other components, e.g., instructions for dilution, mixing and/or administration of the product, other containers, syringes, needles, etc. Other such pack/kit components will be readily apparent to one of skill in the art.

### EXAMPLES

The antineoplastic activity of an mTOR inhibitor plus sunitinib malate combination can be confirmed in in vitro and in vivo standard pharmacological test procedure. The following briefly describes the procedures.

Human rhabdomyosarcoma lines Rh30 and Rh1 and the human glioblastoma line SJ-GBM2 are used for in vitro combination studies with an mTOR inhibitor and sunitinib malate. In vivo studies can use a cell lines from the appropriate neoplasm, e.g., a human neuroblastoma (NB1643), a human colon line GC3, and a human renal cell line.

Dose response curves are determined for each of the drugs of interest. The cell lines, e.g., Rh30, Rh1 and SJ-G2 are plated in six-well cluster plates at 6×10³, 5×10³ and 2.5×10⁴ cells/well respectively. After a 24 hour incubation period, drugs are added in either 10% FBS+RPMI1640 for Rh30 and Rh1 or 15% FBS+DME for SJ-G2. After seven days exposure to drug containing media, the nuclei are released by treating the cells with a hypotonic solution followed by a detergent. The nuclei are then counted with a Coulter Counter. The results of the experiments are graphed and the IC₅₀ (drug concentration producing 50% inhibition of growth) for each drug is determined by extrapolation. Because the IC₅₀s varies slightly from experiment to experiment, two values that bracketed the IC₅₀ of each drug are used in the interaction studies. The point of maximum interaction between two drugs occurs when they are present in a 1:1 ratio if the isobole is of standard shape. Therefore, each of the three approximate IC₅₀ concentrations of an mTOR inhibitor are typically mixed in a 1:1 ratio with each of three approximated IC₅₀s of the sunitinib malate. This results in nine 1:1 combinations of drugs in each experiment plus three IC₅₀ concentrations for mTOR inhibitor and sunitinib malate. This protocol usually results in at least one combination for each drug containing an IC₅₀ value. The 1:1 combination of IC₅₀ concentrations for the mTOR inhibitor and sunitinib malate are then used to calculate additivity, synergism, or antagonism using Berenbaum's formula: x/X₅₀+y/Y₅₀,=1,<1,>1. If the three concentrations of mTOR inhibitor tested alone do not produce an IC that matches any of the three ICs of the sunitinib malate alone, all the 1:1 combinations are checked to see if their ICs fell between the appropriate ICs of drugs tested singly. If they do, the effect was considered additive.

All patents, patent publications, articles, and other documents referenced herein are incorporated by reference. It will be clear to one of skill in the art that modifications can be made to the specific embodiments described herein without departing from the scope of the invention.

1. A method of treating a neoplasm in a mammal in need thereof, which comprises providing to said mammal an effective amount of a combination comprising an mTOR inhibitor and sunitinib malate.

2. The method according to claim 1, wherein the neoplasm is selected from the group consisting of renal cancer, soft tissue cancer, breast cancer, neuroendocrine tumor of the lung, cervical cancer, uterine cancer, head and neck cancer, glioma, non-small lung cell cancer, prostate cancer, pancreatic cancer, lymphoma, melanoma, small cell lung cancer, ovarian cancer, colon cancer, esophageal cancer, gastric cancer, leukemia, colorectal cancer, and unknown primary cancer.

3. The method according to claim 1, wherein said combination further comprises another active component selected from the group consisting of one or more antineoplastic alkylating agent, one or more antimetabolite antineoplastic agents, one or more biochemical immune modulators, imatinib, one or more EGFR inhibitors, a multi-kinase inhibitor that targets serine/threonine and receptor tyrosine kinases in both the tumor cell and tumor vasculature, or an interferon.

4. The method according to claim 3, wherein said combination further comprises one or more antineoplastic agents selected from the group consisting of meclorethamine, cyclophosphamide, ifosfamide, melphalan, chlorambucil, thiotepa, mitomycin, busulfan, lomustine, carmustine, procarbazine, temozolomide, cisplatin, and carboplatin.

5. The method according to claim 3, wherein said combination further comprises an antimetabolite antineoplastic agent selected from the group consisting of: 5-fluorouracil; floxuradine; thioguanine; cytarabine; fludarabine; 6-mercaptopurine; methotrexate; gemcitabine; taxanes; capecitabine; pentostatin; trimetrexatel; and cladribine.

6. The method according to claim 3, wherein said combination further comprises a biochemical modulating agent selected from the group consisting of leucovorin and levofolinate.

7. The method according to claim 3, wherein the combination further comprises an interferon.

8. The method according to claim 7, wherein the interferon is selected from interferon α, interferon β, and interferon γ.

9. The method according to claim 1, wherein either the mTOR inhibitor, sunitinib malate, or both are provided in subtherapeutically effective amounts.

10. The method according to claim 1, wherein the mTOR inhibitor is rapamycin.

11. The method according to claim 1, wherein the mTOR inhibitor is 42-O-(2-hydroxy)ethyl rapamycin.

12. A method of treating renal cell carcinoma in a mammal in need thereof, which comprises providing to said mammal an effective amount of a combination comprising temsirolimus and sunitinib malate.

13. The method according to claim 12, wherein either temsirolimus or sunitinib malate, or both are provided in subtherapeutically effective amounts.

14. The method according to claim 13, wherein either temsirolimus or sunitinib malate is provided in a supratherapeutic dose.

15. The method according to claim 12,wherein the method further comprises administering an interferon in combination with temsirolimus and sunitinib malate.

16. A regimen for treatment of renal cell carcinoma, said method comprising:
delivering a dosage amount amount of an mTOR inhibitor weekly; and
delivering a dose of sunitinib malate daily for a period of at least two weeks followed by at least one week off.

17. The regimen according to claim 16, wherein the mTOR inhibitor is delivered intravenously.

18. The regimen according to claim 16, wherein the mTOR inhibitor is delivered orally.

19. The regimen according to claim 16, wherein the sunitinib malate is delivered orally.

20. The regimen according to claim 16, wherein the sunitinib malate is delivered for a period of four weeks followed by two weeks off.

21. The regimen according to claim 16, wherein the mTOR inhibitor is rapamycin or a derivative thereof.

22. The regimen according to claim 21, wherein the rapamycin is temsirolimus.

23. A product containing temsirolimus and sunitinib malate as a combined preparation for simultaneous, separate or sequential use in treating cancer in a mammal.

24. A product containing an mTOR inhibitor and sunitinib malate as a combined preparation for simulatenous, separate or sequential use in treating renal cell carcinoma in a mammal.

25. A pharmaceutical pack containing a course of an anti-neoplastic treatment for one individual mammal, wherein the pack contains (a) at least one unit of temsirolimus and (b) at least one unit of sunitinib malate in unit dosage form.

26. A pharmaceutical pack containing a course of treatment of renal cell carcinoma for one individual mammal, wherein the pack contains (a) at least one unit of an mTOR inhibitor and (b) at least one unit of sunitinib malate in unit dosage form.

27. A pharmaceutical composition useful in treating a neoplasm in a mammal, the composition comprising (a) at least one unit of temsirolimus and (b) at least one unit of sunitinib malate in unit dosage form, and a pharmaceutically acceptable carrier.

28. A pharmaceutical composition useful in treating renal cell carcinoma in a mammal, the composition comprising (a) at least one unit of an mTOR inhibitor and (b) at least one unit of sunitinib malate in unit dosage form, and a pharmaceutically acceptable carrier.
